(19) United States
(12) Patent Application Publication
(10) Pub. No.: US 2007/0067040 A1
(43) Pub. Date: Mar. 22, 2007

(54) Methods and apparatus for reconstructing the anulus fibrosus

(75) Inventor: Bret A. Ferree, Cincinnati, OH (US)

Correspondence Address: O'MELVENY & MYERS LLP, 610 NEWPORT CENTER DRIVE, 17TH FLOOR, NEWPORT BEACH, CA, 92660, US

(73) Assignee: Anova Corporation

(21) Appl. No.: 11/601,395

(22) Filed: Nov. 17, 2006

Related U.S. Application Data
(63) Continuation-in-part of application No. 11/514,506, filed on Sep. 1, 2006.
(60) Provisional application No. 60/713,969, filed on Sep. 2, 2005.
(60) Provisional application No. 60/738,833, filed on Nov. 21, 2005.

(51) Int. Cl. A61F 2/44; A61B 17/80

(52) U.S. Cl. 623/17.16; 606/69

(57) ABSTRACT

Methods and devices for fixing a defect in the anulus fibrosus of a patient. The devices include first and second vertical components extending from the middle region of the horizontal component, each of the first and second vertical components having a width and an end. The middle region of the horizontal component of the device blocks the defect in the anulus fibrosus. The first vertical component is attached to the upper vertebra and the second vertical component is attached to the lower vertebra. The horizontal component can be positioned beneath a layer of the posterior longitudinal ligament.

This is a continuation-in-part of U.S. application Ser. No. _{11/514,506}, filed Nov. 1, 2005, which claims the benefit of U.S. Provisional Patent Application Ser. No. 60/713,969, filed Sep. 2, 2005. This also claims the benefit of U.S. Provisional Patent Application Ser. No. 60/738,833, filed Nov. 21, 2005. All of the above-referenced applications are hereby expressly incorporated by reference herein in their entirety.

### REFERENCE TO RELATED APPLICATIONS

This application is also related to U.S. application Ser. No. 11/187,250, filed Jul. 22, 2005, which claims priority from U.S. Provisional Patent Application Ser. No. 60/590,942, filed Jul. 23, 2004.

U.S. patent application Ser. No. 11/187,250 is a continuation-in-part of U.S. patent application Ser. No. 10/120,763, filed Apr. 11, 2002, which is a continuation-in-part of U.S. patent application Ser. No. 09/807,820, filed Apr. 19, 2001, now abandoned, which is a U.S. national phase application of PCT/US00/14708, filed May 30, 2000; and Ser. No. 09/638,241, filed Aug. 14, 2000; and Ser. No. 09/454,908, filed Dec. 3, 1999, now U.S. Pat. Nos. 6,491,724; and 09/639,309, filed Aug. 14, 2000, now U.S. Pat. Nos. 6,419,702; and 09/690,536, filed Oct. 16, 2000, now U.S. Pat. No. 6,371,990, which is a continuation-in-part of U.S. patent application Ser. No. 09/638,726, filed Aug. 14, 2000, now U.S. Pat. No. 6,340,369; and Ser. No. 09/415,382, filed Oct. 8, 1999, now U.S. Pat. No. 6,419,704.

U.S. patent application Ser. No. 11/187,250 is also a continuation-in-part of U.S. patent application Ser. No. 10/185,284, filed Jun. 26, 2002, which is a continuation-in-part of U.S. patent application Ser. Nos. 10/120,763, filed Apr. 11, 2002; Ser. No. 09/807,820, filed Apr. 19, 2001, now abandoned; and Ser. No. 09/415,382, filed Oct. 8, 1999, now U.S. Pat. No. 6,419,704, and Ser. No. 10/191,639, filed Jul. 9, 2002.

U.S. patent application Ser. No. 11/187,250 is also a continuation-in-part of U.S. patent application Ser. No. 10/303,385, filed Nov. 25, 2002, which is a continuation-in-part of U.S. patent application Ser. No. 09/415,382, filed Oct. 8, 1999, now U.S. Pat. No. 6,419,704, and Ser. No. 10/191,639, filed Jul. 9, 2002.

U.S. patent application Ser. No. 11/187,250 is also a continuation-in-part of U.S. patent application Ser. No. 10/991,733, filed Nov. 18, 2004, which is a continuation-in-part of U.S. patent application Ser. No. 10/421,434, filed April 23, which claims priority from U.S. Provisional Patent Application Ser. Nos. 60/375,185, filed Apr. 24, 2002 and 60/378,132, filed May 15, 2002. The entire content of each application and patent is incorporated herein by reference.

### BACKGROUND OF THE INVENTION

The human intervertebral disc is an oval to kidney bean-shaped structure of variable size depending on the location in the spine. The outer portion of the disc is known as the anulus fibrosus (AF, also known as the “annulus fibrosis”). The anulus fibrosus is formed of approximately 10 to 60 fibrous bands or layers. The fibers in the bands alternate their direction of orientation by about 30 degrees between each band. The orientation serves to control vertebral motion (one half of the bands tighten to check motion when the vertebra above or below the disc are turned in either direction).

The anulus fibrosus contains the nucleus pulposus (NP). The nucleus pulposus serves to transmit and dampen axial loads. A high water content (approximately 70-80%) assists the nucleus in this function. The water content has a diurnal variation. The nucleus imbibes water while a person lies recumbent. Nuclear material removed from the body and placed into water will imbibe water swelling to several times its normal size. Activity squeezes fluid from the disc. The nucleus comprises roughly 50% of the entire disc. The nucleus contains cells (chondrocytes and fibrocytes) and proteoglycans (chondroitin sulfate and keratin sulfate). The cell density in the nucleus is on the order of 4,000 cells per microliter.

The intervertebral disc changes or “degenerates” with age. As a person ages, the water content of the disc falls from approximately 85% at birth to approximately 70% in the elderly. The ratio of chondroitin sulfate to keratin sulfate decreases with age, while the ratio of chondroitin 6 sulfate to chondroitin 4 sulfate increases with age. The distinction between the annulus and the nucleus decreases with age. Generally disc degeneration is painless.

Premature or accelerated disc degeneration is known as degenerative disc disease. A large portion of patients suffering from chronic low back pain are thought to have this condition. As the disc degenerates, the nucleus and annulus functions are compromised. The nucleus becomes thinner and less able to handle compression loads. The annulus fibers become redundant as the nucleus shrinks. The redundant annular fibers are less effective in controlling vertebral motion. This disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments for disc degeneration are destructive. One group of procedures, which includes lumbar discectomy, removes the nucleus or a portion of the nucleus. A second group of procedures destroy nuclear material. This group includes Chymopapin (an enzyme) injection, laser discectomy, and thermal therapy (heat treatment to denature proteins). The first two groups of procedures compromise the treated disc. A third group, which includes spinal fusion procedures, either remove the disc or the disc's function by connecting two or more vertebra together with bone. Fusion procedures transmit additional stress to the adjacent discs, which results in premature disc degeneration of the adjacent discs. These destructive procedures lead to acceleration of disc degeneration.

Prosthetic disc replacement offers many advantages. The prosthetic disc attempts to eliminate a patient's pain while preserving the disc's function. Current prosthetic disc implants either replace the nucleus or replace both the nucleus and the annulus. Both types of current procedures remove the degenerated disc component to allow room for the prosthetic component. Although the use of resilient materials has been proposed, the need remains for further improvements in the way in which prosthetic components are incorporated into the disc space to ensure strength and longevity. Such improvements are necessary, since the prosthesis may be subjected to 100,000,000 compression cycles over the life of the implant.

Current nucleus replacements (NRs) may cause lower back pain if too much pressure is applied to the anulus fibrosus. As discussed in co-pending U.S. patent application Ser. No. 10/407,554 and U.S. Pat. No. 6,878,167, the content of each being expressly incorporated herein by reference in their entirety, the posterior portion of the anulus fibrosus has abundant pain fibers.

Herniated nucleus pulposus (HNP) occurs from tears in the anulus fibrosus. The herniated nucleus pulposus often allies pressure on the nerves or spinal cord. Compressed nerves cause back and leg or arm pain. Although a patient's symptoms result primarily from pressure by the nucleus pulposus, the primary pathology lies in the anulus fibrosus.

Surgery for herniated nucleus pulposus, known as microlumbar discectomy (MLD), only addresses the nucleus pulposus. The opening in the anulus fibrosus is enlarged during surgery, further weakening the anulus fibrosus. Surgeons also remove generous amounts of the nucleus pulposus to reduce the risk of extruding additional pieces of nucleus pulposus through the defect in the anulus fibrosus. Although microlumbar discectomy decreases or eliminates a patient's leg or arm pain, the procedure damages weakened discs.

### SUMMARY OF THE INVENTION

The invention broadly facilitates reconstruction of the anulus fibrosus (AF) and the nucleus pulposus (NP). Such reconstruction prevents recurrent herniation following microlumbar discectomy. The invention may also be used in the treatment of herniated discs, annular tears of the disc, or disc degeneration, while enabling surgeons to preserve the contained nucleus pulposus. The methods and apparatus may be used to treat discs throughout the spine including the cervical, thoracic, and lumbar spines of humans and animals.

The invention also enables surgeons to reconstruct the anulus fibrosus and replace or augment the nucleus pulposus. Novel nucleus replacements (NR) may be added to the disc. Annulus reconstruction prevents extrusion of the nucleus replacements through holes in the anulus fibrosus. The nucleus replacements and the anulus fibrosus reconstruction prevent excessive pressure on the anulus fibrosus that may cause back or leg pain. The nucleus replacements may be made of natural or synthetic materials.

Synthetic nucleus replacements may be made of, but are not limited to, polymers including polyurethane, silicon, hydrogel, or other elastomers.

In the preferred embodiment, a spinal repair system according to the invention comprises a first end portion adapted for placement within an intervertebral body, a second end portion adapted for placement within an adjacent intervertebral body, and a bridge portion connecting the first and second end portions, the bridge portion being adapted to span a portion of an intervertebral disc space and prevent excessive outward bulging.

The first and second end portions may be composed of a rigid biocompatible material, including metals, alloys, or ceramics, and the bridge portion is composed of a flexible, braided or mesh material. Preferably, however, the first and second end portions are composed of allograft bone and the bridge portion is composed of allograft anulus fibrosus. A single piece of allograft tissue, such as fascia, may alternatively be used. The system may further include screws and/or plates to hold the first and second end portions into respective vertebral bodies.

In one configuration the first and second end portions are elongate, and the bridge portion spans the end portions in a plane parallel to the end portions. The system may further comprise slotted bone dowels into which the end portions are received, and the bridge portion extends through one slot and into the other when implanted. The system may further include an artificial disc replacement (ADR) defining a volume, with the bridge portion extending through at least a portion of the volume of the ADR.

The end and bridge portions may together form a cylindrical shape. At least one of the end portions may be threaded. One or both of the end portions may be configured for bony ingrowth. Various instruments and methods are also disclosed.

A spinal repair method according to the invention includes the steps of forming a first hole or channel in a first intervertebral body, placing the first end portion into the first hole or channel, forming a second hole or channel in an adjacent intervertebral body, and placing the second end portion into the second hole or channel such that the bridge portion spans a hole or defect in an anulus fibrosus. The end portions may then be secured with screws. The step of providing the system may include harvesting the portions from a human or animal donor, with the end portions comprising intervertebral bone and the bridge portion comprises anulus fibrosus still attached to the end portions.

Although drawings illustrate use of the invention in the lumbar spine, the invention may also be used in other portions of the body. For example, the invention may be used to reconstruct the anterior portion of the cervical spine, the knee, or other joints of the body.

In an alternative embodiment, the invention provides a device that includes a horizontal component having first and second ends, a middle region, and a length. The device also includes first and second vertical components extending from the middle region of the horizontal component, each of the first and second vertical components having a width and an end. In one embodiment, the length of the horizontal component of the device is longer than the width of each of the first and second vertical components. The device may be in the form of a plus (“+”) sign or a cross. The device may be made from allograft soft tissue, polypropylene, polytetrafluoroethylene, polyester, polyethylene terephthalate, or other appropriate biocompatible materials.

In an alternative embodiment, the invention provides a method for fixing a defect in the anulus fibrosus of an intervertebral disc of a patient, the intervertebral disc being located between an upper and a lower vertebrae. A device is provided that includes a horizontal component having first and second ends, a middle region, and a length. The device also includes first and second vertical components extending from the middle region of the horizontal component, each of the first and second vertical components having a width and an end. In one embodiment, the length of the horizontal component of the device is longer than the width of each of the first and second vertical components. The middle region of the horizontal component of the device to block the defect in the anulus fibrosus. The first vertical component is attached to the upper vertebra and the second vertical component is attached to the lower vertebra.

The horizontal component can be positioned beyond at least an outer layer of the anulus fibrosus, alternatively positioned beyond the innermost layer of anulus fibrosus, alternatively positioned between adjacent layers of anulus fibrosus, or alternatively positioned on the exterior of the anulus fibrosus. The horizontal component may be attached to the anulus fibrosus with at least one fixation device, such as a staple. Alternatively, the horizontal component may be attached to the anulus fibrosus on either side of the defect with multiple fixation devices.

The method may also include locating a growth promoting component within the defect. The growth-promoting component may be made from allograft tissue, xenograft tissue, collagen-soaked BMP sponges, or autograft material. The allograft tissue may be fascia, tendon, or anulus fibrosus. The xenograft tissue may be porcine intestinal sub-mucosa.

In an alternative embodiment, the invention provides a device with multiple horizontal arms or components. The device includes a vertical component comprising an upper and a lower region and first, second, third, and fourth horizontal components extending from the vertical component. The device may be made from allograft soft tissue, polypropylene, polytetrafluoroethylene, polyester, or polyethylene terephthalate.

In an alternative embodiment, the invention provides a method for fixing a defect in the anulus fibrosus of an intervertebral disc of a patient, the intervertebral disc being located between an upper and a lower vertebrae with a device with two sets of horizontal arms or components. A device is provided that includes a vertical component having an upper, middle, and lower region, and first, second, third, and fourth horizontal components extending from the vertical component. The middle region of the vertical component is positioned to block the defect in the anulus fibrosus. The first horizontal component is positioned behind an innermost layer of the anulus fibrosus on the right side of the defect. The second horizontal component is positioned in front of an outermost layer of the anulus fibrosus on the right side of the defect. The third horizontal component is positioned behind an innermost layer of the anulus fibrosus on the left side of the defect. The second horizontal component is positioned in front of an outermost layer of the anulus fibrosus on the left side of the defect. The upper region of the vertical component is attached to the upper vertebra. The lower region of the vertical component to the lower vertebra.

In an alternative embodiment, the invention provides a method for fixing a defect in the anulus fibrosus of an intervertebral disc of a patient, the intervertebral disc being located between an upper and a lower vertebrae using a device that is secured to the vertebrae with a fixation material. A device is provided that includes a horizontal component having first and second ends, a middle region, and a length. The device also includes first and second vertical components extending from the middle region of the horizontal component, each of the first and second vertical components having a width and an end. In one embodiment, the length of the horizontal component of the device is longer than the width of each of the first and second vertical components. The horizontal component of the device is positioned to block the defect in the anulus fibrosus. The first vertical component is inserted into a hole in the upper vertebra. The second vertical component is inserted into a hole in the lower vertebra. A fixation material is injected into the hole in the upper vertebra. The fixation material may be an in-situ curing polymer. In an alternative method, the horizontal component may also be attached to the anulus fibrosus.

The fixation material may be injected into the vertebrae with an injection tool that has an enlarged distal end that is capable of substantially occluding the hole in which the material is being injected. The enlarged distal end may be an inflatable bladder or a deformable element.

In an alternative embodiment, the invention provides a device that includes a multiple layers and has an upper region, a middle region, and a lower region. The upper and lower regions each have two layers of a material and the middle region has three layers of the material. The device also includes a securing or binding element secured around the middle region of the multilayered device. The device may be made from allograft soft tissue, polypropylene, polytetrafluoroethylene, polyester, and polyethylene terephthalate. The device may be made from a single sheet of material of multiple sheets of material.

In the preferred embodiments, surgical mesh devices are placed between the anulus fibrosus and the posterior longitudinal ligament (PLL), or between the posterior longitudinal ligament and the periosteum that lies over the vertebrae, or both. The device and the location of the device optimize tissue ingrowth into the device yet minimize the risk of adhesions between the device and the nerves within the spinal canal.

The device is placed over the outside of the anulus fibrosus, the portion of the anulus fibrosus with the highest healing potential. The device is placed anterior to the PLL and/or periosteum to prevent tissue (adhesions) from forming between the disc and the nerves. The PLL also prevents the device from eroding into the nerves.

The devices are preferably constructed from the materials that promote tissue ingrowth. For example, polypropylene or polyester surgical meshes may be used to construct the devices.

### BRIEF DESCRIPTION OF THE FIGURES

FIG. 1 illustrates a flexible implant.

FIG. 2A illustrates a sagittal cross-section of the spine with the flexible implant.

FIG. 2B illustrates a sagittal cross-section of the spine with the flexible implant with bone ingrowth into the vertebral holes.

FIG. 3A illustrates a posterior aspect of the spine with the flexible implant of the current invention with the vertical arms secured to the surrounding vertebrae with interference screws.

FIG. 3B illustrates an axial cross-section of a disc and the embodiment of the invention positioned beyond the innermost layer of the anulus fibrosus.

FIG. 4A illustrates a posterior aspect of the spine with the flexible implant of the current invention with the vertical arms secured to the surrounding vertebrae with interference screws.

FIG. 4B illustrates an axial cross-section of a disc and the embodiment of the invention attached to the outer layer of the anulus fibrosus.

FIG. 5 illustrates an axial cross-section of a disc and an alternative embodiment of the current invention with a growth-promoting component positioned within the defect.

FIG. 6 illustrates an axial cross-section of a disc and an alternative flexible implant with two horizontal arms.

FIG. 7 illustrates an alternative embodiment of the current invention with cylindrical structures attached to the horizontal arms.

FIG. 8 illustrates an insertion tool to be used with the device of FIG. 7.

FIG. 9A illustrates the insertion tool of FIG. 8 inserted into the device of FIG. 7.

FIG. 9B illustrates an axial view of a disc and the embodiments of FIGS. 7 and 8.

FIG. 10 illustrates an alternative embodiment of the device, which has openings in the horizontal arms.

FIG. 11A illustrates an axial cross section of a disc with the flexible device of FIG. 10 inserted in the disc.

FIG. 11B illustrates a sagittal cross section of a portion of the embodiments of the invention in FIG. 11A.

FIG. 12A illustrates an alternative embodiment of the device, which has openings in the vertical arms.

FIG. 12B illustrates a sagittal cross section of a portion of the embodiments of the invention in FIG. 12A.

FIG. 13 illustrates an alternative embodiment of the device with reinforced areas in the vertical arms.

FIG. 14A illustrates an alternative embodiment of the device with bladders in the vertical arms.

FIG. 14B illustrates an alternative embodiment of the device with the bladders in the vertical arms inflated.

FIG. 15A illustrates an alternative embodiment of the device with openings in the vertical arms.

FIG. 15B illustrates an alternative embodiment of the device with mesh-like sections in the vertical arms.

FIG. 16A illustrates an alternative embodiment of the device with pockets in the vertical arms.

FIG. 16B illustrates an anterior view of the embodiment of the invention of FIG. 16A and an insertion tool.

FIG. 16C illustrates a sagittal cross section of a portion of the spine and the embodiment of the invention of FIG. 16B.

FIG. 16D illustrates an anterior view of the embodiment of the invention of FIG. 16A and an alternative insertion tool.

FIG. 16E illustrates a sagittal cross section of the spine and the embodiment of the invention drawn in FIG. 16A.

FIG. 16F illustrates a sagittal cross section of the spine and the embodiment of the invention drawn in FIG. 16A with in-situ curing polymer inserted into the holes in the vertebrae.

FIG. 17 illustrates an alternative embodiment of the device with alternative vertical components.

FIG. 18A illustrates an alternative multi-layered embodiment of the device.

FIG. 18B is a lateral view of the embodiment of FIG. 18A.

FIG. 18C is a sagittal cross-section of the spine and the embodiment of the invention drawn in FIG. 18B.

FIG. 19A illustrates an alternative embodiment of the device having two separate components.

FIG. 19B illustrates a posterior view of the spine with the embodiment of FIG. 19A.

FIG. 20A illustrates bone-growth promoting plugs.

FIG. 20B illustrates the embodiments of FIG. 20A inserted into the holes in the vertebrae.

FIG. 21A illustrates a sagittal cross-section of an interference screw.

FIG. 21B illustrates a sagittal cross-section of an alternative interference screw.

FIG. 22A illustrates a sagittal cross-section of a spine and injection of a fixation material.

FIG. 22B illustrates a sagittal cross-section of a spine and the embodiment of the invention drawn in FIG. 22A.

FIG. 23A illustrates a sagittal cross-section of a spine and a polymer injection tool.

FIG. 23B illustrates a sagittal cross-section of a spine and an alternative polymer injection tool.

FIG. 24 illustrates an alternative polymer injection tool.

FIG. 25A illustrates a posterior view of a sagittal cross-section of a portion of the lumbar spine.

FIG. 25B illustrates a sagittal cross section of two lumbar vertebrae and their associated ligaments.

FIG. 25C illustrates an axial cross section of lumbar spine at the disc level.

FIG. 26 illustrates a posterior view of an alternative embodiment of the invention having vertical and horizontal arms.

FIG. 27 illustrates is a lateral view of a sagittal cross-section of the spine and the embodiment of the invention drawn in FIG. 26.

FIG. 28A illustrates an axial cross-section of a disc and the embodiment of the invention drawn in FIG. 26.

FIG. 28B illustrates is another axial cross section of a disc and the embodiment of the invention drawn in FIG. 26.

FIG. 29 illustrates is an axial cross-section of a disc and an alternative embodiment of the invention having horizontal and vertical arms and an anti-adhesion component.

FIG. 30 illustrates is an axial cross section of a disc and an exploded view of an alternative embodiment of the invention having horizontal and vertical arms, an anti-adhesion component, and a component extending into the defect.

FIG. 31 illustrates is an axial cross section of a disc and device 240 of FIG. 30 with alternate fixation members connecting the device to the anulus fibrosus.

FIG. 32A illustrates is posterior view of an alternative embodiment of the invention having a cover component and a cylindrical component for insertion into the defect.

FIG. 32B illustrates an anterior view of the device of FIG. 32A.

FIG. 32C illustrates a sagittal cross-section of the spine and the embodiment of the invention drawn in FIG. 32B.

FIG. 33A illustrates is a sagittal cross section of an alternative embodiment of the invention inserted into the humerus.

FIG. 33B illustrates is a sagittal cross-section of the humerus and the embodiment of the invention drawn in FIG. 33A.

FIG. 33C illustrates a lateral view of the end of the invention drawn in FIG. 33B.

FIG. 33D illustrates an enlarged view of a partial sagittal cross section of humerus and the embodiment of the invention drawn in FIG. 33A.

FIG. 34A illustrates a lateral view of the end of an alternative embodiment of the invention wherein one end of the suture is made of mesh.

FIG. 34B illustrates a lateral view of a partial sagittal cross-section of the humerus and the embodiment of the invention drawn in FIG. 34A.

### DESCRIPTION OF THE INVENTION

Anatomy

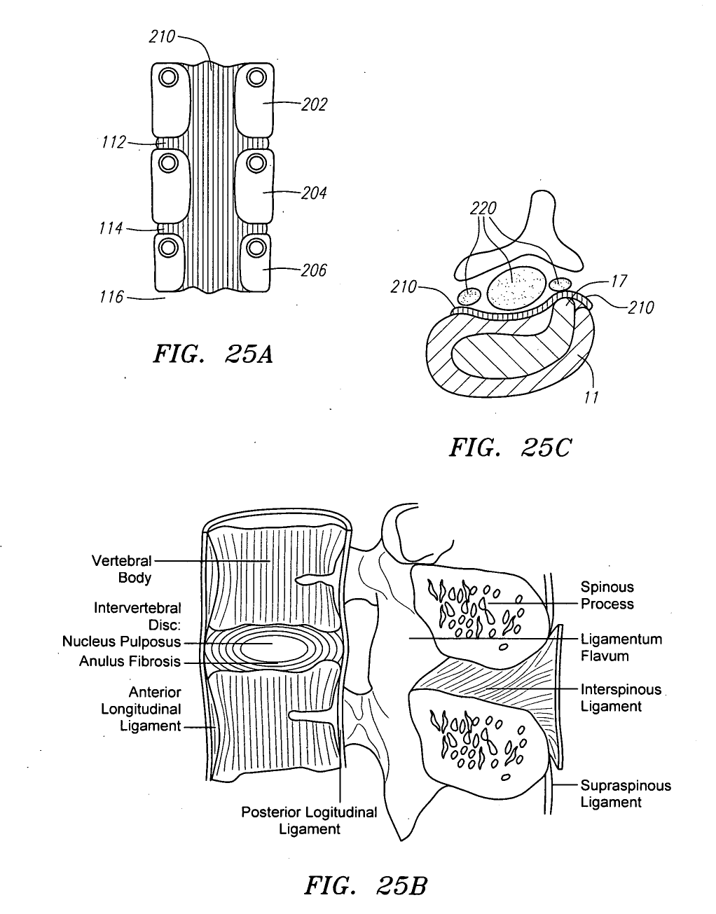

FIG. 25A is a posterior view of a sagittal cross-section through a portion of the lumbar spine. The drawing shows bisected pedicles 202, 204, 206 of three lumbar vertebrae. Posterior longitudinal ligament (PLL) 210 courses over the middle of the vertebrae and fans out over the posterior portions 112, 114, 116 of the intervertebral discs. FIG. 25B is a sagittal cross section of two lumbar vertebrae and their associated ligaments. Posterior longitudinal ligament (PLL) 210 can be seen covering the posterior side of the intervertebral disc. It lies between the discs and the thecal sac and exiting nerves. The thecal sac also contains spinal nerves. Posterior longitudinal ligament (PLL) 210 is loosely attached to the anulus fibrosus of the discs and likely contributes cells that grow into devices placed adjacent to itself. Posterior longitudinal ligament (PLL) 210 also prevents adhesions between the thecal sac/nerves and any device that is placed between posterior longitudinal ligament (PLL) 210 and the disc.

FIG. 25C is an axial cross section of lumbar spine at the disc level. The drawing shows herniation of nucleus pulposus 17 through a defect in the anulus fibrosus 11. Posterior longitudinal ligament (PLL) 210 lies between both anulus fibrosus 11, the herniated portion of nucleus pulposus 17, and nerves 220.

Implant Devices

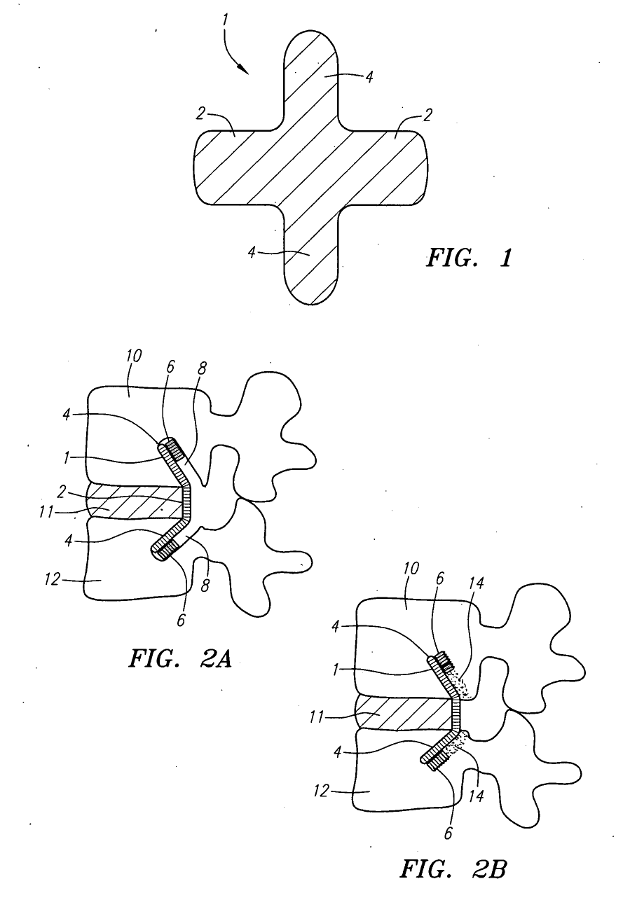

FIG. 1 is a posterior view of an embodiment of the current invention. The flexible device 1 has a pair of horizontal arms 2 and vertical arms 4. Device 1 may be made of allograft soft tissue, such as fascia. Alternatively, device 1 may be made of a synthetic material. For example, a woven mesh of polypropylene, expanded polytetrafluoroethylene (PTFE, Gortex), polyester (e.g. Dacron, DuPont Wilmington, Del.), polyethylene terephthalate (PET) or other bio-compatible polymeric films or fibers may be used. The polymeric films or fibers may be biaxially oriented. In one embodiment, the material may have a burst strength of about 20-150 psi, alternatively of about 50-120 psi. In an alternative embodiment, device 1 may be about 10-60 mm tall, about 5-50 mm wide, and about 0.05-15 mm thick. Vertical arms 4 of the device may be about 4-25 mm tall and about 1-20 mm wide. Alternatively, vertical arms 4 may be about 2-8 mm wide and about 10-20 mm long. Horizontal arms 2 of the device may be about 5-50 mm in length and about 1-20 mm tall.

FIG. 2A is a sagittal cross section of the spine and the embodiment of the invention drawn in FIG. 1. Device 1 is anchored into upper and lower vertebrae 10, 12 and covers a portion of the outside of intervertebral disc, i.e., the outermost layer of anulus fibrosus 11. Device 1 is held in the spine by interference screws 6 inserted into upper and lower vertebrae 10, 12. Interference screws 6 are countersunk into the holes 8 drilled into the vertebrae. Alternatively, the interference screws may be flush with the surface of the vertebrae (not shown). Flush placement of the screws enables the screws to press against the cortical walls of the vertebrae.

FIG. 2B is a sagittal cross section of the spine and the embodiment of the invention drawn in FIG. 2A. The patient's bone 14 has grown into holes 8 in upper and lower vertebrae 10, 12. Bone growth into the holes helps prevent extrusion of device 1. In one embodiment, flexible vertical arms 4 preferably have holes that permit the patient's bone to grow through the flexible arms. Bone growth through the portion of the device within the bone tunnels helps stabilize the device.

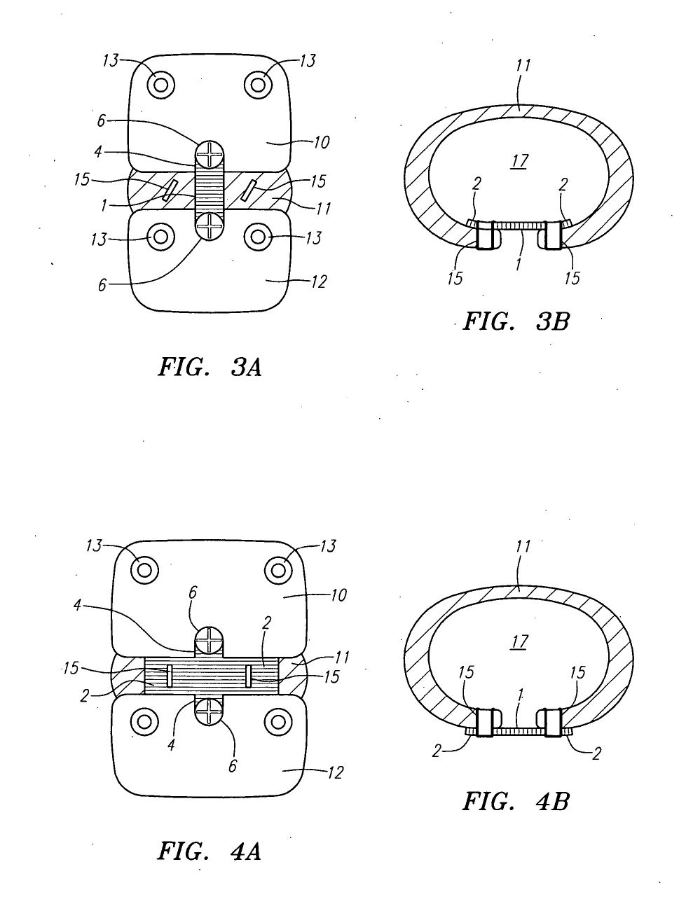

FIG. 3A is a view of the posterior aspect of the spine and the embodiment of the invention drawn in FIG. 2A. The posterior elements of vertebrae 10, 12 have been removed to improve the view of device 1. The sets of circles 13 in each vertebra represent the cross section of bisected pedicles of the vertebrae. Vertical arms 4 of device 1 are shown spanning anulus fibrosus 11 and attached to upper and lower vertebrae 10, 12. The heads of interference screws 6 are seen stabilizing the device in upper and lower vertebrae 10, 12. Anchors, such as staples 15, pass through the anulus fibrosus and into horizontal arms of the device (not shown), which are positioned beyond at least the outermost layer of the anulus fibrosus 11. Alternative fixation devices may be used in a manner similar to staples 15. For example, sutures may be passed through the annulus and the device with suture passing instruments used in shoulder arthroscopy procedures.

FIG. 3B is an axial cross section of a disc and the embodiment of the invention. Horizontal arms 2 may be disposed beyond at least the outer layer of the anulus fibrosus 11. Horizontal arms may be disposed between layers of anulus fibrosus 11. Horizontal arms 2 may alternatively be disposed past the innermost layer of anulus fibrosus 11 and lie between the anulus fibrosus and the nucleus pulposus 17 located in the intradiscal space, as shown in FIG. 3B. Staples 15 pass through the anulus fibrosus and horizontal arms 2 of device 1.

FIG. 4A is a view of the posterior aspect of a bisected spine and an alternative embodiment of the invention. Horizontal arms 2 of device 1 have been positioned on the outside of the intervertebral disc and are attached to the exterior of the anulus fibrosus 111 with staples 15 in regions of the anulus fibrosus near, adjacent to, or surrounding the defect in the anulus fibrosus.

FIG. 4B is an axial cross section of a disc and the embodiment of the invention. Horizontal arms 2 of device 1 are shown positioned on the outside of the intervertebral disc and are attached to the exterior of the anulus fibrosus 11 with staples 15.

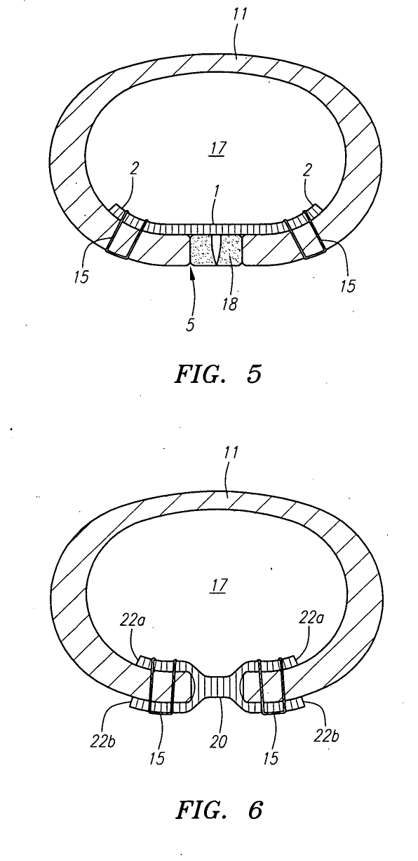

FIG. 5 is an axial cross section of a disc and an alternative embodiment of the invention in which a composite device is used to repair the defect in the anulus fibrosus. For example, a growth promoting material 18 may be attached to or associated with device 1. The growth promoting material 18 may loosely fit into the aperture 5 in the anulus fibrosus 11. The loose fit between the growth promoting component 18 and the walls of the aperture 5 of the anulus fibrosus permit fluids, cells, or other materials to pass into and out of the disc. Movement of fluids and cells into and out of the disc may facilitate healing of the disc. The growth promoting component 18 could be made of allograft tissue such as fascia, tendon, or anulus fibrosus; xenograft tissue such as porcine intestinal sub-mucosa; collagen-soaked BMP sponges; or autograft material. Alternatively, the composite device may be made of two or more different types of allograft tissue. For example, an allograft anulus fibrosus component or meniscus component may be attached to an allograft fascial component. The growth promoting material 18 may be added to any embodiment of the device, regardless of whether the horizontal arms are positioned on the inside, outside, or within the layers of the anulus fibrosus.

Modifications to Horizontal Arms

FIG. 6 is an axial cross section of a disc 11 and an alternative embodiment of the invention. Device 20 has two sets of horizontal arms 22a, 22b that are anchored to regions of the anulus fibrosus near, adjacent to, or surrounding the defect in the anulus fibrosus. The first set of arms 22a are placed on the interior of the anulus fibrosus 11. The second set of arms 22b are placed on the exterior of the anulus fibrosus 11. Fixation devices or anchors, such as staples 15, are attached to each of set of the horizontal anchors 22a, 22b and extend through the anulus fibrosus 11.

FIG. 7 is an oblique view of an alternative embodiment the device. Horizontal arms 2 of the device have tube-like openings or cylindrical structure 25 at the ends of the horizontal arms. At least one cylindrical structure 25 having a lumen between the proximal and distal ends of each cylindrical structure is located on each horizontal arm to add in placement and positioning of the device. In one embodiment, the device has at least two cylindrical structures 25 located on each horizontal arm, preferably near the end of each horizontal arm 2. The lumens of cylindrical structures 25 act as a female joint and are capable of receiving a prong of an insertion tool within the lumen.

FIG. 8 is an oblique view of an insertion tool. Prongs 35 extending from the distal end of insertion tool 30 are designed to fit into the lumens of the cylindrical structures 25 in the embodiment of the invention drawn in FIG. 7. It is understood that the number of prongs at the distal end of the insertion tool will match the number of cylindrical structures located on each horizontal arm. For example, in the embodiment where there is only one cylindrical structure on the end of each horizontal arm, there will only be one prong at the end of the insertion tool.

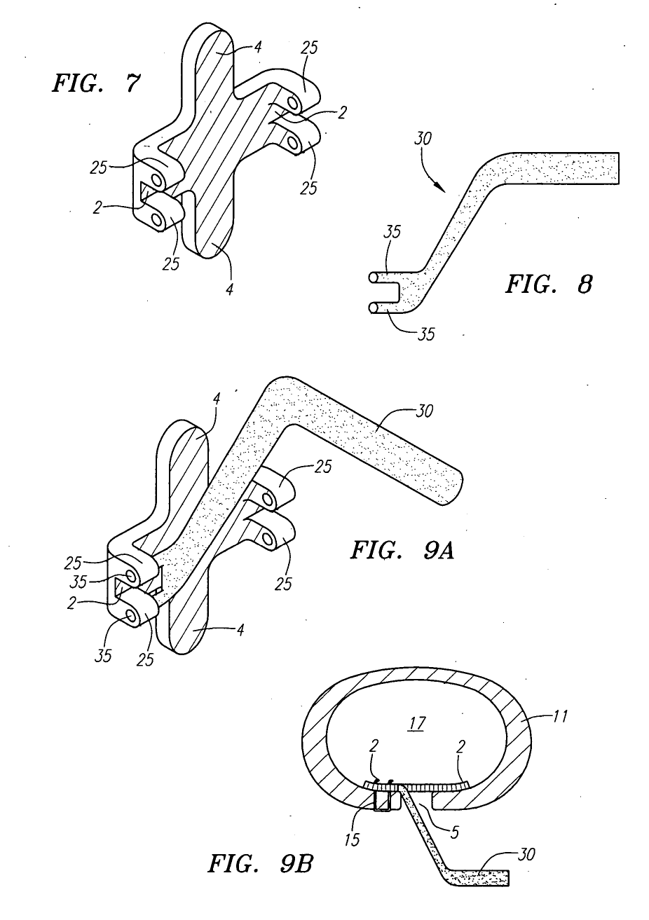

FIG. 9A is an oblique view of the embodiments of the invention drawn in FIGS. 7 and 8. Insertion tool 30 has been inserted into the device, where prongs 35 have been inserted into the lumens of cylindrical structures 25 located on horizontal arms 2.

FIG. 9B is an axial view of a disc and the embodiments of the invention drawn in FIGS. 7 and 8. Insertion tool 30 passes through the opening 5 in the anulus fibrosus 11. Insertion tool 30 may be used to position horizontal arms 2 of the device against the anulus fibrosus 11. As seen in FIG. 9B, horizontal arms 2 are being positioned against the innermost layer of anulus fibrosus 11. Horizontal arms 2 of the device may be attached or anchored to the anulus fibrosus 11. For example, horizontal arms 2 of the device may attached or anchored to the interior of the anulus fibrosus 11 through staples 15 that extend from the exterior to the interior of anulus fibrosus 11, including horizontal arms 2. Attaching horizontal arms 2 of the device to the anulus fibrosus 11 helps prevent the escape of intradiscal material, such as nucleus pulposus 17, nucleus replacement, or intradiscal devices between the device and the anulus fibrosus 11. The vertical arms of the device and the interference screws inserted into the surrounding vertebrae cooperate to hold the device within or such that the device blocks the opening in anulus fibrosus.

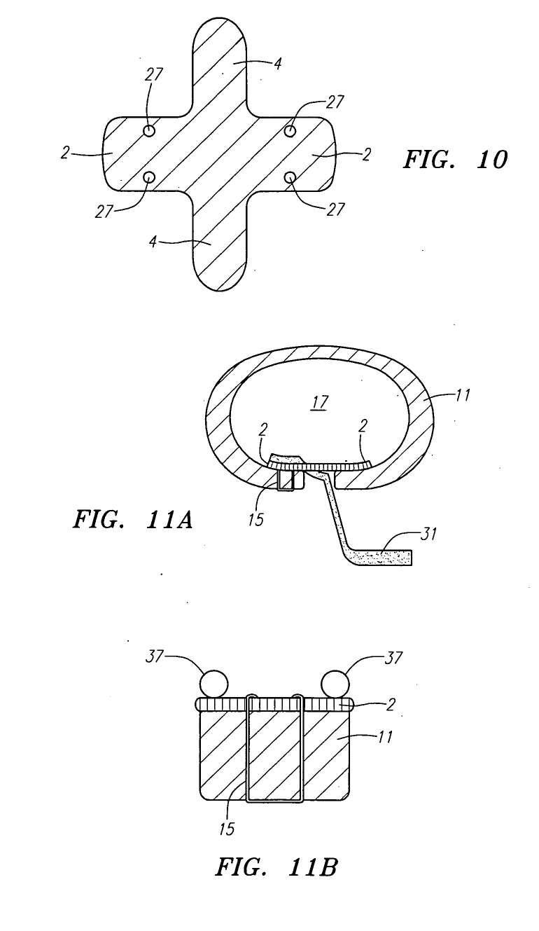

FIG. 10 is an anterior view of an alternative embodiment. Horizontal arms 2 of the device contain holes 27. Each horizontal arm 2 may contain one hole, alternatively two holes, alternatively three holes, alternatively four holes, alternatively five or more holes. The holes are capable adapted to receive prongs 37 located at the distal end of an insertion tool 31. It is understood that the number of prongs at the distal end of the insertion tool will match the number of holes located on each horizontal arm. For example, in the embodiment where there are two holes located on each horizontal arm, there will only be two prongs at the end of the insertion tool. Preferably, the prongs will extend in a perpendicular direction from a longitudinal axis of the distal region of the insertion tool. The holes may optionally be surrounded by reinforcing components (not shown).

FIG. 11A is an axial cross section of a disc, the embodiment of the inventions drawn in FIGS. 10 and 11. Prongs 37 of the tool pass through holes 27 in horizontal arms 2. Insertion tool 31 positions horizontal arms 2 of the device against the interior of anulus fibrosus 11.

FIG. 11B is sagittal cross section of a portion of a disc and a portion of the embodiments of the invention drawn in FIG. 11A. Arms of staple 15 or other fixation member may pass in the region of the anulus fibrosus and horizontal arm between holes 27 and prongs 37 of insertion tool 31 located in the holes.

Modifications to Vertical Arms

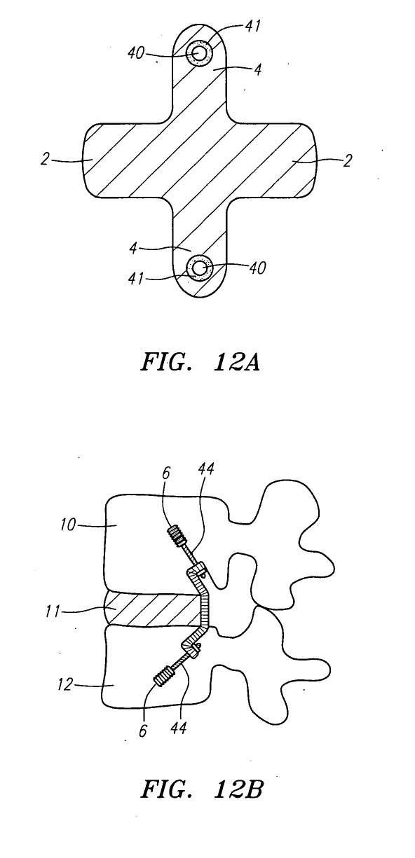

FIG. 12A is an anterior view of an alternative embodiment of the invention. Vertical arms 4 of the device contain holes 40. Holes 40 may be surrounded by reinforcing components 41, such as grommets.

FIG. 12B is a sagittal cross section of the spine and the embodiment of the invention drawn in FIG. 12A. Suture anchors 44 pass through holes 40 in vertical arms 4 of the device to hold the device in place. Suture anchors 44 may have enlarged proximal and/or distal ends. In one embodiment, crimps have been placed over the cut ends of the sutures. The enlarged proximal end of suture anchor 44 prevents suture anchor 44 from passing through hole 40. The distal end of suture anchor 44 is embedded in the vertebra.

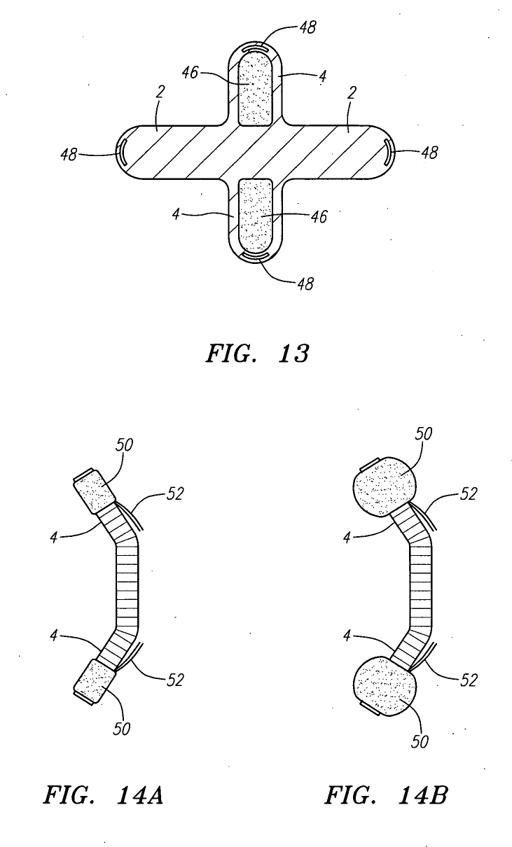

FIG. 13 is an anterior view of an alternative embodiment of the invention. Vertical arms 4 of the device have reinforced areas 46, which helps protect the device as the interference screws are advanced into the vertebrae. Reinforcement may be achieved by using thicker material or, alternatively, a second material. The second material may be impregnated into the mesh or it could be attached to the vertical arms of the device. Reinforcing materials may include bio-compatible polymers, metals, or ceramics. Radiopaque markers 48 may also be placed into the ends of the horizontal and/or vertical arms of the device to aid in visualization during insertion and subsequent examination.

FIG. 14A is a lateral view of an alternative embodiment of the invention. Inflatable bladders 50 may be incorporated into vertical arms 4 of the device. Inflatable bladders 50 are adapted to receive a substance that will secure vertical arms 4 to the upper and lower vertebrae. Inflation lumens 52 are attached to each bladder and communicate with the interior of the bladder.

FIG. 14B is a lateral view of the embodiment of the invention drawn in FIG. 14A. Bladders 50 have been expanded by injecting a substance that will secure the vertical arms to the vertebrae, such as an in-situ curing polymer, through inflation lumens 52. In use, bladders 50 are filled with the substance, such as the polymer, after the device is placed into the spine and the vertical arms are inserted into the surrounding vertebrae. Expansion of bladders 50 locks or secures the device into the vertebrae. Bladder 50 prevents the substance, e.g., polymer monomers, from escaping into the spine and causing any damage.

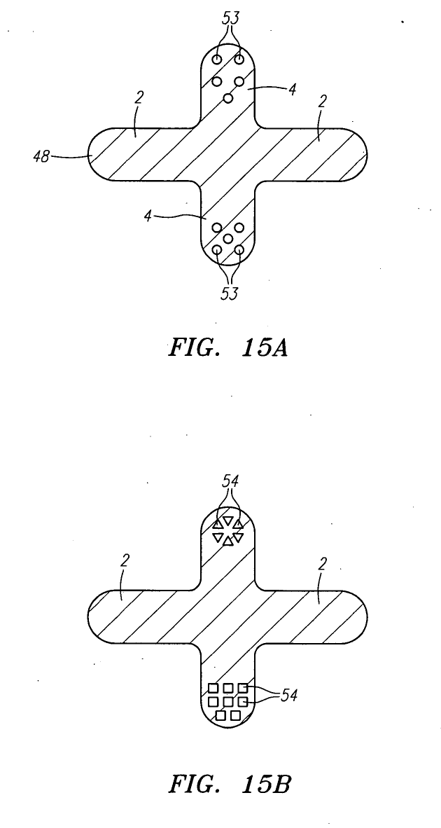

FIG. 15A is an anterior view of an alternative embodiment of the invention. Holes or openings 53 are placed in vertical arms 4 of the device. Holes or openings 53 allow fixation substances, such as in-situ curing polymers, to pass through the device. In an alternative embodiment, the fixation material need not act as an adhesive. The hardened fixation material could act as a grout that holds the vertical components in place by extending through holes or openings in the vertical components and into the cancellous bone of the vertebrae. The holes are particularly useful when the device is made of fascia or other solid material. The holes may be a variety of shapes, including circles, triangles, rectangles, squares, polygons, ellipses. The holes may also be a slit in the material making up the vertical arms. The holes may be about 0.01-2.0 mm in diameter, alternatively about 0.5-1.5 mm in diameter.

FIG. 15B is an anterior view of an alternative embodiment of the invention. A cutting instrument can be used to create mesh-like sections, with openings 54, in vertical arms 4 of the device.

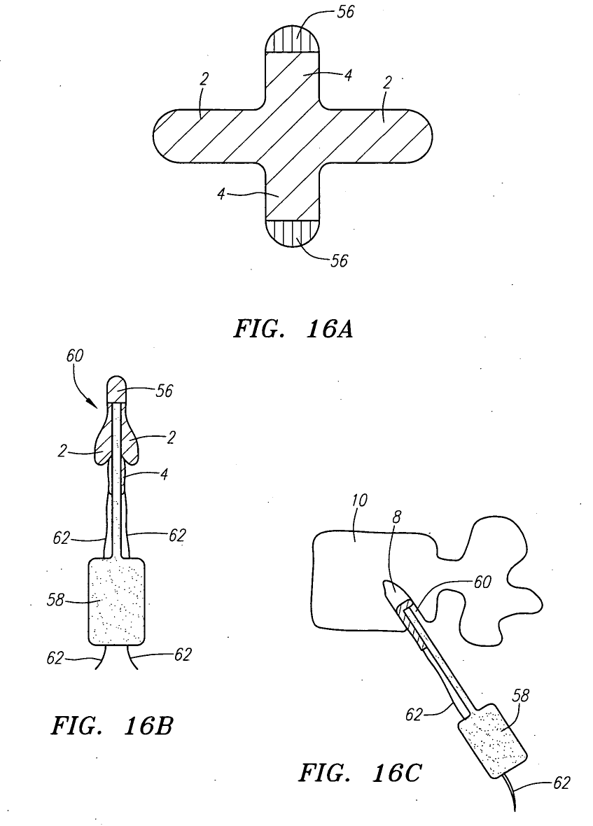

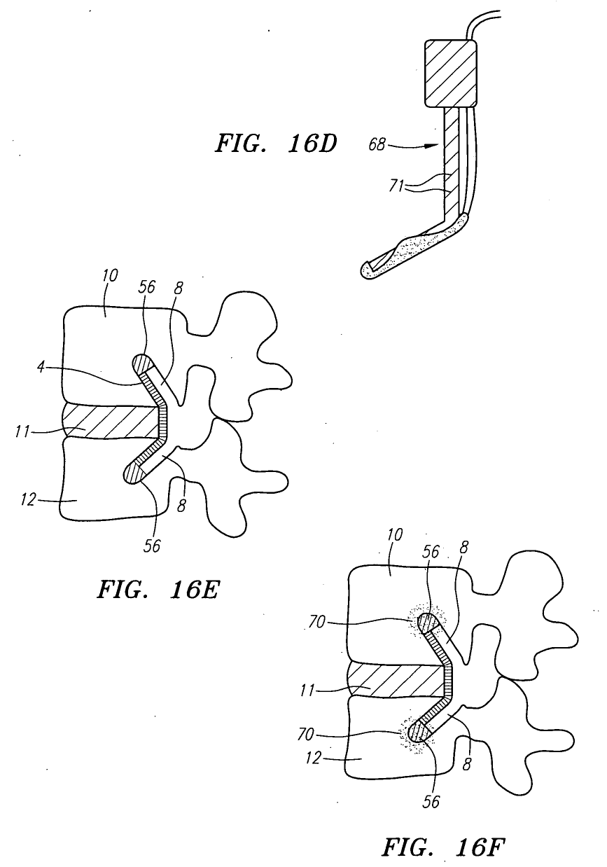

FIG. 16A is an anterior view of an alternative embodiment of the invention. Pockets 56 may be included on the ends, or tips, of vertical arms 4 of the device. Pockets 56 are adapted to receive an insertion tool inserted into the inside of the pocket to facilitate insertion and placement of the vertical arms of the device.

FIG. 16B is an anterior view of the embodiment of the invention drawn in FIG. 16A and an insertion tool 58. The distal end or top 60 of the insertion tool 58 extends into pocket 56 of one of vertical arms 4 of the device. A suture 62 passes from the other end of the device, i.e., the other vertical arm, and through the handle of insertion tool 58. Tension by the cooperation of suture 62 and tool 58 collapse the device along a longitudinal axis of insertion tool 58 to facilitate insertion of the device.

FIG. 16C is a sagittal cross section of a portion of the spine and the embodiment of the invention drawn in FIG. 16B. The collapsed device is inserted into hole 8 in vertebra 10.

FIG. 16D is a lateral view of an alternative embodiment of an insertion tool. The tip of insertion instrument 68 is angled to facilitate insertion of the flexible device into the holes in the vertebrae. Insertion instrument 68 may also be used to inject the polymer or other fixation substance. The tip of the instrument may have marks 71 to indicate the depth that insertion tool 68 has been inserted into the hole. For example, insertion tool 68 could be withdrawn 1 cm after the flexible device has been inserted into the hole. A bladder on the tip of the instrument (not shown) could be inflated to seal the hole after the instrument is withdrawn and the polymer or other fixation substance could be injected after the insertion tool has been partially withdrawn. Markings on the instrument could indicate when the instrument has been withdrawn certain amounts, for example, 1 cm. Fluoroscopy or other navigational devices and techniques may be used to facilitate the procedure. The fixation substance or polymer is preferably radiopaque or has a radiopaque material included in with the polymer, such that the fixation substance can be visualized or otherwise detected by the surgeon. In one method, about 0.25-10 cc of polymer may be injected per hole. Alternatively, about 1-3 cc may be injected per hole.

FIG. 16E is a sagittal cross section of the spine and the embodiment of the invention drawn in FIG. 16A. The enlarged ends of vertical arms 4 containing pockets 56 fill the base of hole 8 in vertebrae 10, 12. The configuration helps hold the device in the spine until the fixation substance, e.g., in-situ curing polymer, is injected. The distal regions or tips of the vertical arms of the device may have barbs, tines, or other features (not shown) to hold and anchor the device in the vertebrae until the polymer is injected.

FIG. 16F is a sagittal cross section of the spine and the embodiment of the invention drawn in FIG. 16E. In-situ curing polymer 70 has been injected into holes 8 in vertebrae 10, 12. Polymer 70 passes through pockets (or pouches) 56 of vertical arms 4 and a portion of the vertical arms of the device. The in-situ curing polymer helps hold the device in the spine.

FIG. 17 is an anterior view of an alternative embodiment of the invention. Vertical arms 4 are configured to increase the device's resistance to extrusion and contain various extensions 74 along the length of the vertical arms. With such a design, the in-situ curing polymer would be able to flow into the creases or voids 75 between extensions 74. Other features may be incorporated into the vertical arms of the device to improve polymers ability to prevent extrusion of the device.

Modifications to Horizontal and Vertical Arms

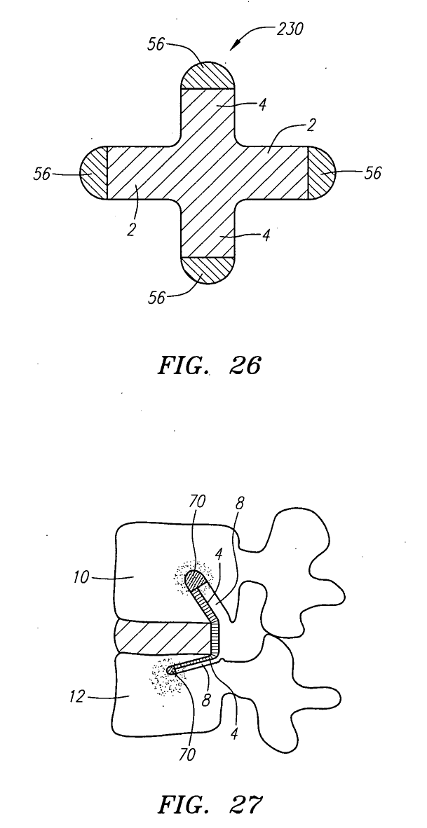

FIG. 26 is a posterior view of an alternative embodiment of the invention. The device 230 has vertical and horizontal arms 4 and 2, respectively. The device may be constructed of polypropylene or polyester surgical mesh. The openings within the mesh are sized to optimize tissue ingrowth. The tips or ends of the horizontal and vertical arms have pockets 56. As described with respect to FIG. 16, the tip of a tool may be placed into each pocket to direct the device between the anulus fibrosus and the posterior longitudinal ligament.

FIG. 27 is a lateral view of a sagittal cross-section of the spine and the embodiment of the invention drawn in FIG. 26. Vertical arms 4 of the device are located in holes 8 drilled into the vertebrae 10 and 12. The device is fastened to vertebrae 10 and 12 with an in-situ curing material 70 such as polymethylmethacrylate (PMMA) or a bioactive cement.

FIG. 28A is an axial cross-section of a disc and the embodiment of the invention drawn in FIG. 26. The device 230 lies between the posterior longitudinal ligament 210 and the anulus fibrosus 11. Horizontal arms 2 of the device extend medial and lateral to defect 5 in the anulus fibrosus 11. Posterior longitudinal ligament 210 and/or periosteum cover the posterior portion of device 230 and prevent adhesions from forming on the posterior side of the device.

FIG. 28B is an axial cross section of a disc and the embodiment of the invention drawn in FIG. 26, but wherein the posterior longitudinal ligament 210 covers only a portion of device 230. Adhesions may form in the uncovered portion 231 where the nerves (not shown) lie against the device.

Alternative Device Configurations

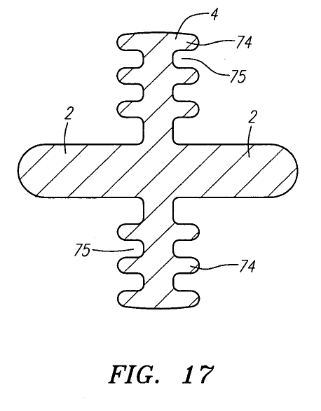

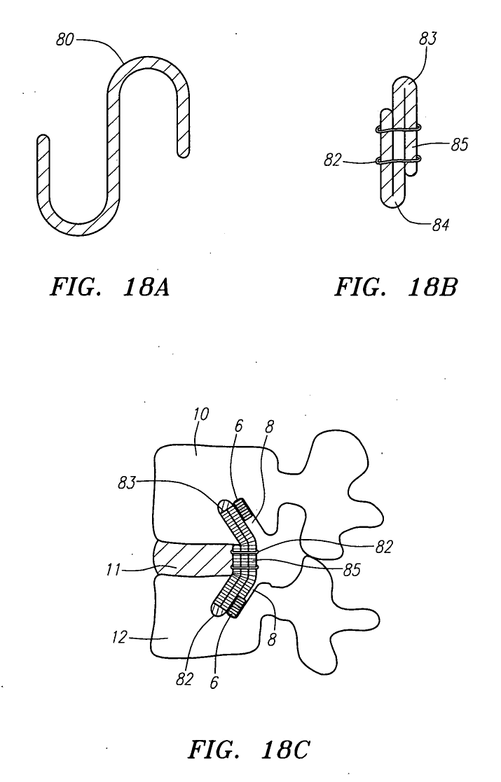

FIG. 18A is a lateral view of an alternative embodiment of the invention. The flexible device may be made of a single piece of material 80 capable of being folded at least twice to form a multi-layered flexible implant. The flexible device may be made of allograft soft tissue, such as fascia. Alternatively, the flexible device may be made of a synthetic material. For example, a woven mesh of polypropylene, expanded polytetrafluoroethylene (PTFE, cortex), polyester (e.g. Dacron, DuPont Wilmington, Del.), polyethylene terephthalate (PET) or other bio-compatible polymeric films or fibers may be used. The polymeric films or fibers may be biaxially oriented.

FIG. 18B is a lateral view of the embodiment of the invention drawn in FIG. 18A. Multi-layered flexible device 80 has been folded and fixed in its folded position using a suture or tie 82. Multi-layered flexible device 80 has been folded to form an upper region 83 and a lower region 84 that are capable of being attached to the upper and lower vertebra, respectively. As seen in FIG. 18B, upper and lower regions 83, 84 have fewer layers of material than the thicker middle section 85 that is to be positioned to over the defect in the anulus fibrosus. The embodiment of this invention may help strengthen devices made of allograft or other materials.

FIG. 18C is a sagittal cross section of the spine and the embodiment of the invention drawn in FIG. 18B. Multi-layered flexible device 80 has been positioned in the spine such that middle section 85 is positioned over the defect in anulus fibrosus 11 and upper and lower regions 83, 84 have been inserted into holes 6 in upper and lower vertebrae 10, 12, respectively. Upper and lower regions 83, 84 are anchored to upper and lower vertebrae 10, 12 using interference screws 6 inserted into holes 8.

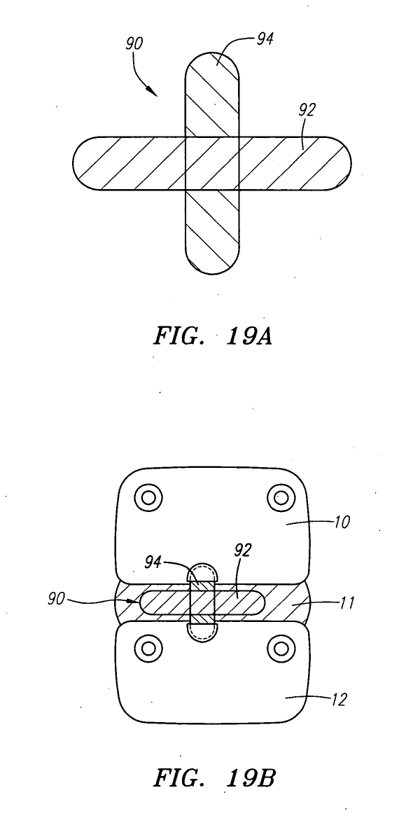

FIG. 19A is an anterior view of an alternative embodiment. Flexible device 90 is constructed from two or more materials making up horizontal component 92 and vertical component 94. For example, vertical component 94 may be made of polymers or another material with high tensile strength. Vertical Component 94 may be attached to horizontal component 92. Horizontal component 92 may have a lower tensile strength and be made of allograft or xenograft tissue or softer polymeric material with lower tensile strength.

FIG. 19B is a posterior view of a portion of the spine and the embodiment of the invention drawn in FIG. 19A. The posterior elements of the spine have been removed to improve the view of the posterior aspect of the disc. Vertical member 94 of device 90 has been fastened to upper and lower vertebrae 10, 12. Horizontal component 92 has been fastened to vertical component 94 and/or the anulus fibrosus 11 on either side of the defect in the anulus fibrosus.

FIG. 29 is an axial cross-section of a disc and an alternative embodiment of the invention. Horizontal arms 2 of the device extend medial and lateral to defect 5 in the anulus fibrosus 11. Vertical arms (not shown) can be attached to the surrounding vertebrae. Composite device 235 has anti-adhesion component 244 that fills the space between the sides of posterior longitudinal ligament 210. Anti-adhesion component 244 is made of materials that are unlikely to form adhesions with the nerves, such as Gortex, autograft fascia, allograft fascia, Seprafilm (Genzyme Corporation, Cambridge Mass.), carboxymethylcellulose, hyaluronic acid, oxidized atelocollagen type I, polyethylene glycol, glycerol, Coseal (Baxter), Tisseal (Baxter), Floseal (Baxter), Duragen Plus (LifeSciences Corporation), or combinations of the materials. Staples 15 fasten the horizontal arms 2 of the device and posterior longitudinal ligament 210 to the anulus fibrosus 11.

FIG. 30 is an axial cross section of a disc and an exploded view of an alternative embodiment of the invention. Horizontal arms 2 of the device extend medial and lateral to defect 5 in the anulus fibrosus 11. Vertical arms (not shown) can be attached to the surrounding vertebrae. Device 240 also has a component 242 that is adapted to extend into defect 5 in anulus fibrosus 11. Component 242 that extends into the defect is constructed of material that promotes tissue ingrowth, such as polypropylene or polyester surgical mesh. Posterior longitudinal ligament 210 has been sectioned and partially elevated from anulus fibrosus 11. Similar to the device described in FIG. 29, device 240 also has anti-adhesion component 244 that is adapted to fill the space between the sides of posterior longitudinal ligament 210. Staples 15 can fasten the horizontal arms 2 of the device and posterior longitudinal ligament 210 to the anulus fibrosus 11.

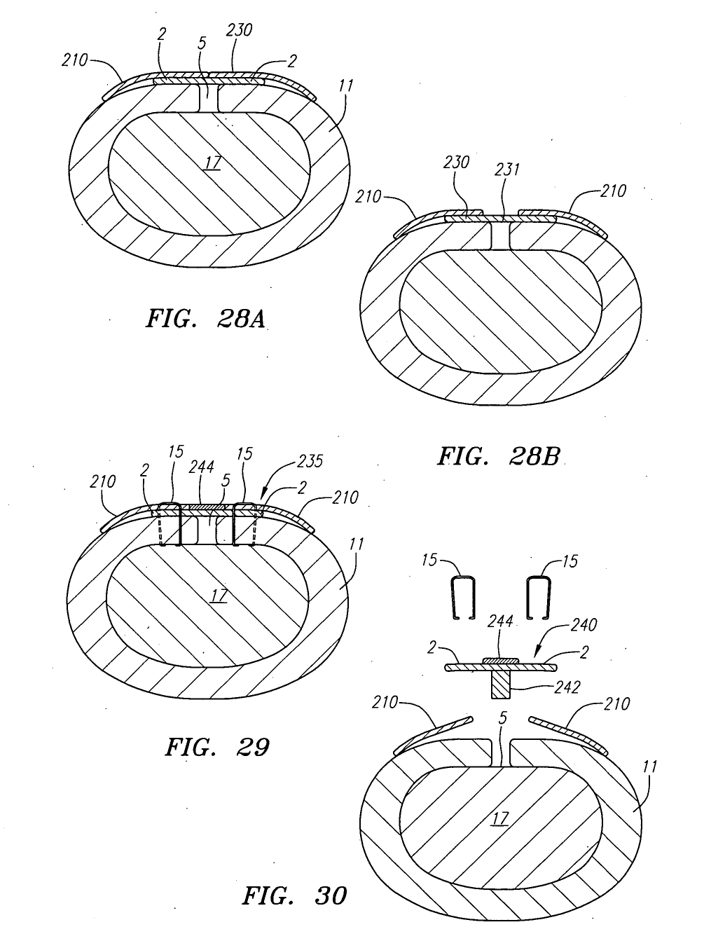

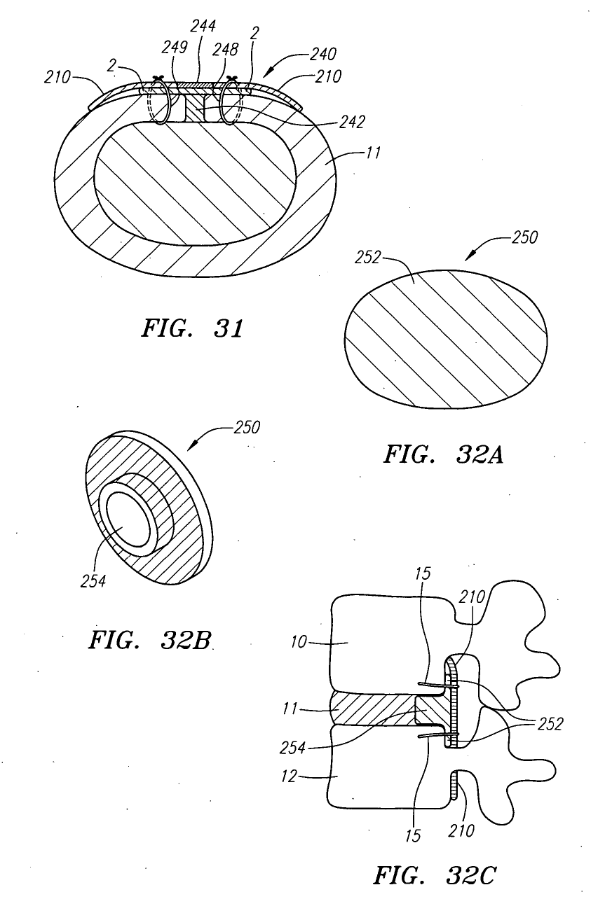

FIG. 31 is an axial cross section of a disc and device 240 of FIG. 30 with alternate fixation members connecting the device to the anulus fibrosus. Horizontal arms 2 of device 240 are fastened to anulus fibrosus 11 with suture 249 on one side of the defect and with a flexible cord 248 having enlargements at either end on the other side of the defect. The enlargements at the ends of the flexible cords may be transverse elements that are substantially perpendicular to the axis of the flexible cord. Alternatively, the enlargements at the ends of the flexible cords may be spherical or otherwise bulbous enlargement. Alternative fastening mechanisms may also be used to hold the device against the anulus fibrosus until tissues grow into the device.

FIG. 32A is posterior view of an alternative embodiment of the invention, and FIG. 32B is an anterior view. Device 250 has cover component 252 and cylindrical component 254 that protrudes from the anterior side of cover component 252. Cover component 252 is sized such that a portion overlaps with the anulus fibrosis on either side of the defect and with the surrounding vertebrae above and below the defect. Cylindrical component 254 is configured for placement in the opening in the anulus fibrosus.

FIG. 32C is a sagittal cross-section of the spine and the embodiment of the invention drawn in FIG. 32B. Cover component 252 of device 250 lies between the anulus fibrosus 111 and posterior longitudinal ligament 210 and/or the periosteum. Posterior longitudinal ligament 210 and/or the periosteum prevent adhesions from forming on the posterior side of cover component 252. Cylindrical component 254 of device 250 also extends into the defect in anulus fibrosus 11. Device 250 is fastened to the vertebrae 10, 12 with staples 15. The arms of staples 15 are preferably designed to course in non-parallel directions as staples 15 are impacted into vertebrae 10, 12. For example, the tips of staples 15 could be shaped to force the arms of staples 15 to diverge as the staple is forced into the vertebrae. Device 250 can also be attached to anulus fibrosus 11 with staples. Alternative fastening devices, including devices made of resorbable materials, can be used to attach the device to the disc or the vertebrae.

Device 250 may be made of allograft soft tissue, such as fascia. Alternatively, device 250 may be made of a synthetic material. For example, a woven mesh of polypropylene, expanded polytetrafluoroethylene (PTFE, Gortex), polyester (e.g. Dacron, DuPont Wilmington, Del.), polyethylene terephthalate (PET) or other bio-compatible polymeric films or fibers may be used. The polymeric films or fibers may be biaxially oriented. In one embodiment, the material may have a burst strength of about 20-150 psi, alternatively of about 50-120 psi. In an alternative embodiment, device 1 may be about 10-60 mm tall, about 5-50 mm wide, and about 0.05-15 mm thick. Cover component 252 may extend about 10-20 mm vertically above and below the defect in the anulus fibrosus to overlap with the surrounding vertebrae. Cover component 252 may extend about 5-50 mm horizontally on either side of the defect to overlap with the surrounding anulus fibrosus.

Fixation Members

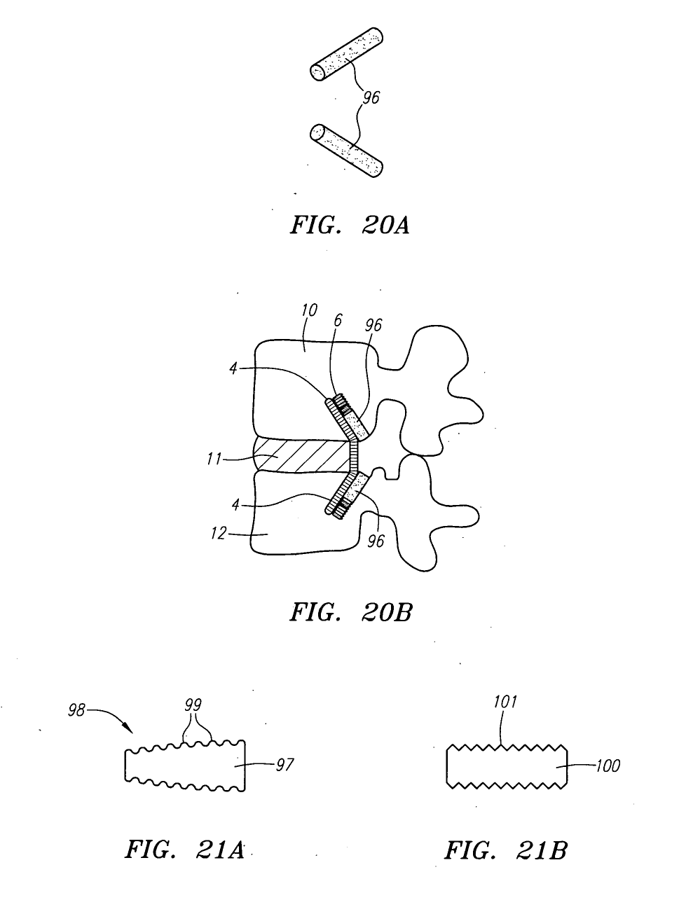

FIG. 20A is an oblique view of plugs 96 that are bone growth promoting dowel shaped devices. Plugs 96 may be made of allograft bone, hydroxyapatite, ceramic, BMP soaked collagen sponges, or other material that promotes or facilitates bone ingrowth.

FIG. 20B is a sagittal cross section of the spine and the embodiments of the invention drawn in FIGS. 2A and 20A. Plugs 96 have been placed into the holes drilled into upper and lower vertebrae 10, 12. Plugs 96 accelerate stabilization of the flexible device by helping to secure vertical arms 4 while also helping to prevent bleeding from the holes in the vertebrae.

FIG. 21A is a sagittal cross section of interference screw 97. The leading edge 98 of screw 97 is slightly tapered to facilitate advancement of screw 97. Interference screws press fit the vertical arms of the device against vertebrae. The edges of the threads 99 of screw 97 are rounded to help prevent damage to the vertical arms of the device, which may be damaged as the interference screws are advanced into the holes in the vertebrae. The interference screws may be cannulated (not shown), i.e., contain a lumen extending from a proximal end to a distal end of the screw. Cannulated screws may be passed over K-wires.

The interference screws are designed to fit into the holes in the vertebrae. The holes drilled into the vertebrae may be approximately 1-15 mm in diameter and approximately 1-30 mm deep. In one embodiment, the holes are approximately 2-3 mm in diameter and approximately 12-25 mm deep. The interference screws may be approximately 2-12 mm in diameter and approximately 5-30 mm long, alternatively approximately 4-6 mm in diameter and approximately 10-20 mm long. The interference screws may be made of titanium or other bio-compatible metal. Alternatively the screws may be made of bioresorbable materials. Alternatively, the interference screws may be made of bone or other bio-active materials including fully cured polymers listed above.

FIG. 21B is a sagittal cross section of an alternative interference screw 100. The ends of the threads 101 are tapered. Tapered threads may be preferred in certain embodiments of the invention because the sharp edges of the tapered screws help the screws cut into and hold pieces of bone. For example, interference screws with tapered threads may be preferred in embodiments of the invention similar to that drawn in FIG. 13A of co-pending U.S. application Ser. No. 11/187,250, which depicts a device that includes a piece of donor anulus fibrosus sandwiched by pieces of donor vertebra on either side. The bone pieces are preferably about 2 to about 16 mm in diameter and about 5 to about 35 mm in length. The anulus fibrosus piece is preferably about 2 to about 40 mm wide and about 5 to about 20 mm tall. The allograft anulus fibrosus could be cylindrical in shape. In one embodiment, the implant device is about 7-8 mm in diameter and the allograft anulus fibrosus is about 8-16 mm long and the bone components are about 10-15 mm long.

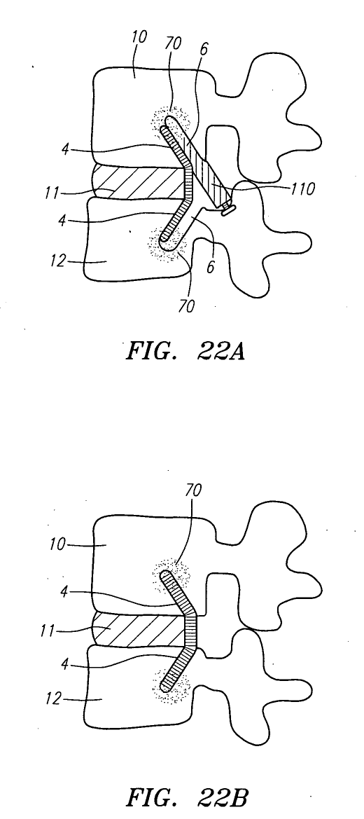

FIG. 22A is a sagittal cross section of the spine and illustrates injection of a curing material or polymer into the hole in the vertebra. Vertical arms 4 of the flexible device are held in place with an in-situ curing polymer 70. Polymer 70 is forced through holes or pores within the vertical arms that are made of mesh, allograft, or flexible member and into the cancellous bone of the vertebrae. The cured polymer locks the vertical arms of the flexible device within the vertebrae. Suitable polymers include polymethylmethacrylate (PMMA), bioactive “cements” such as calcium phosphate, hydroxyapatite, carbonated apatite cement, and glass-ceramic powders. Other biocompatible in-situ curing materials may be used such as polyurethane, hydrogel, or bio-active glues.

Polymer delivery vehicle 110 preferably temporarily seals hole 6 in the vertebra. Sealing hole 6 prevents extrusion of polymer 70 into the spinal canal. Sealing hole 6 also enables pressurization of the polymer to facilitate passage of the polymer into the vertebrae and through the holes or pores within the vertical arms. A small portion of hole 6 is preferably left open to allow bone in-growth, or to pack bone growth promoting materials, such as the plug described in FIG. 20. In fact, the vertical arms of the flexible device could be attached or fastened to the vertebra by impacting pieces of bone, including allograft bone, ceramic or other material into the holes in the vertebrae. Alternatively, the interference screws used in other embodiments of the invention could be made of bone or other bio-active materials including fully cured polymers listed above.

FIG. 22B is a sagittal cross section of the spine and the embodiment of the invention drawn in FIG. 22A. Bone has grown into the holes in the vertebrae. Bone ingrowth further stabilizes the implant. Bone may also grow into, partially replace, or fully replace, bioactive materials, or resorbable materials used to temporarily stabilize the mesh device. Some of the polymer may remain in the vertebrae, with bone ingrowth in the proximal portion of the holes in some embodiments of the device. Preferred resorbable materials are listed in co-pending applications included by reference in this application.

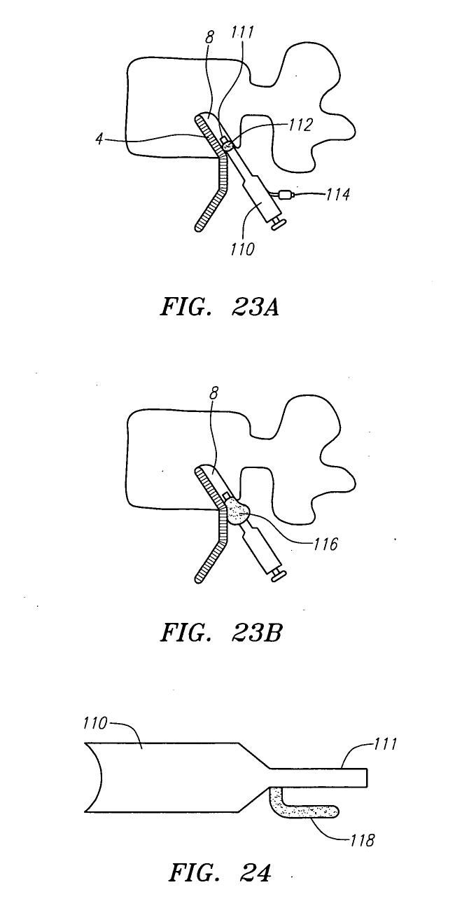

FIG. 23A is a sagittal cross section of a portion of the spine and an alternative polymer injection tool 110. Polymer injection tool 110 has an inflatable bladder 112 near or at its distal end 111. Inflatable bladder 112 is inflated after the tip of the polymer injection tool 110 is placed into hole 8 of the vertebra. In use, inflating bladder 112 forms a temporary seal between polymer injection tool 110 and the vertebra. Inflatable bladder 112 is deflated after the polymer is at least partially cured, enabling polymer injection tool 110 or a catheter (not shown) attached to polymer injection tool 110 to be removed from the spine. Port 114 on the side of polymer injection tool 110 or catheter may be used to inflate and deflate bladder 112.

FIG. 23B is a sagittal cross section of a portion of the spine and an alternative embodiment of the invention drawn in FIG. 23A. The tip, distal end, or distal region of polymer injection tool 110 or a catheter attached to polymer injection tool 110 includes a deformable element 116. Polymer injection tool 110 or catheter may by press fit into holes 6 in the vertebrae, thus forming a temporary seal. Other polymer injection delivery systems may be used in the invention.

FIG. 24 is a lateral view of a portion of the tip of an alternative polymer delivery tool. Injection tool 110 has projection 118 that may be used to increase the tension on the vertical arms of the device before and while the polymer is injected. Projection 118 is adapted to fit into an opening or hole located in the vertical arms of the implanted device. For example, the projection may fit into a hole in a mesh device.

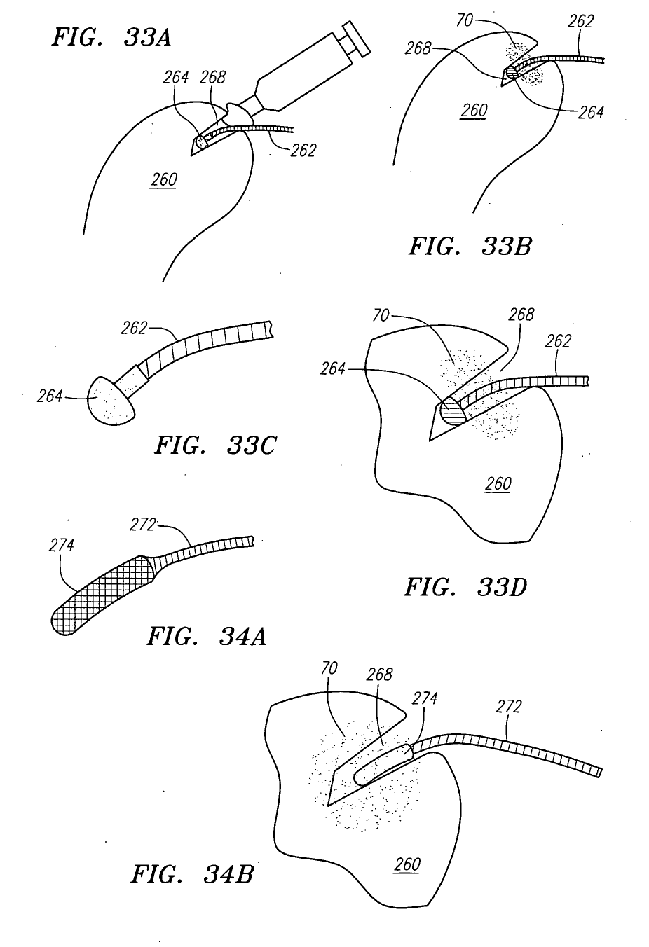

FIGS. 33A through 33B represent alternative embodiments of the invention useful in the spine as well as other joints and bony tissue. FIG. 33A is a sagittal cross section through the humerus 260, showing a suture 262 with an enlarged end 264 placed into hole 268 drilled into the humeral head. An in-situ curing material 10, such as Polymethylmethacrylate (PMMA) or a bio-active cement is injected into hole 268 in FIG. 33B.

FIG. 33B is a sagittal cross-section of humerus 260 and the embodiment of the invention drawn in FIG. 33A. In-situ curing material 70 was forced into the cancellous bone surrounding hole 268. Enlarged end 264 of suture 262 is trapped beyond the fully cured material 70, thus fastening suture 262 to the bone. The invention may be used in other bones such as the tibia, femur, fibula or other bones of the body in a similar manner.

FIG. 33C is a lateral view of the end of the invention drawn in FIG. 33B. Enlarged end 264 of suture 902 is preferably press-fit into hole 268, provisionally securing the device until the PMMA or other in-situ curing material 70 is injected. FIG. 33D is an enlarged view of a partial sagittal cross section of humerus 260 and the embodiment of the invention drawn in FIG. 33A. The enlarged end of the suture is trapped distal to the cured material.

FIG. 34A is a lateral view of the end of an alternative embodiment of the invention wherein one end of suture 272 is made of mesh 274. Alternatively, the end of the suture that is fastened to the bone may have holes. FIG. 34B is a lateral view of a partial sagittal cross-section of humerus 260 and the embodiment of the invention drawn in FIG. 34A. PMMA or other in-situ curing material 70 has been injected through the holes of mesh 274 and into the bone surrounding hole 268 in humerus 260.

Methods of Implantation into the Spine

The devices described above can be inserted in various places with respect to the spine. The devices may be inserted between the posterior longitudinal ligament (PLL) and the anulus fibrosus (AF), between the vertebrae and the posterior longitudinal ligament (PLL), between the anulus fibrosus (AF) and the anterior longitudinal ligament (ALL), between the anterior longitudinal ligament (ALL) and the vertebrae, between layers of the anterior longitudinal ligament (ALL), between layers of the posterior longitudinal ligament (PLL), between layers of the anulus fibrosus (AF), or under other tendons or ligaments that can contribute cells to the mesh and act as a barrier to adhesions between the mesh and the tissue that lie over a second side of the tendon, ligament, anulus fibrosus, or other tissue.

In one embodiment, the device is inserted between the posterior longitudinal ligament (PLL) and the anulus fibrosus (AF). This method enables the posterior longitudinal ligament to contribute cells to the mesh and prevents adhesions between the mesh and the nerves. A slit or multiple slits may be made in the posterior longitudinal ligament adjacent the injured disc. The horizontal arms of the mesh device can then be inserted beneath the posterior longitudinal ligament. This may be accomplished using an instrument similar to that described in FIGS. 16B-D. Alternatively, sutures attached to the device may be first threaded under the posterior longitudinal ligament on either side of the aperture in the anulus fibrosus. The free-ends of the sutures may then be pulled to position the device between the posterior longitudinal ligament and the anulus fibrosus. The horizontal arms of the device may then be attached to the anulus fibrosus on either side of the aperture using anchors, such as staples, that pass through both the horizontal arms and the anulus fibrosus. Alternatively, sutures or other fixation devices may be used to attach the horizontal arms to the anulus fibrosus. The vertical arms are attached to the upper and lower vertebrae using interference screws that are countersunk into holes that are drilled into the vertebrae. The interference screws may be inserted such that they are flush with the surface of the vertebrae, which enables the screws to press against the cortical walls of the vertebrae. In one method, the vertical arms can also be placed in a similar manner between the vertebrae and the posterior longitudinal ligament (PLL).

In one embodiment, the device is inserted between the anterior longitudinal ligament (ALL) and the anulus fibrosus (AF). This method enables the anterior longitudinal ligament to contribute cells to the mesh and prevents adhesions between the mesh and the nerves. A slit or multiple slits may be made in the anterior longitudinal ligament adjacent the injured disc. The horizontal arms of the mesh device can then be inserted beneath the anterior longitudinal ligament. This may be accomplished using an instrument similar to that described in FIGS. 16B-D. Alternatively, sutures attached to the device may be first threaded under the anterior longitudinal ligament on either side of the aperture in the anulus fibrosus. The free-ends of the sutures may then be pulled to position the device between the anterior longitudinal ligament and the anulus fibrosus. The horizontal arms of the device may then be attached to the anulus fibrosus on either side of the aperture using anchors, such as staples, that pass through both the horizontal arms and the anulus fibrosus. Alternatively, sutures or other fixation devices may be used to attach the horizontal arms to the anulus fibrosus. The vertical arms are attached to the upper and lower vertebrae using interference screws that are countersunk into holes that are drilled into the vertebrae. The interference screws may be inserted such that they are flush with the surface of the vertebrae, which enables the screws to press against the cortical walls of the vertebrae. In one method, the vertical arms can also be placed in a similar manner between the vertebrae and the anterior longitudinal ligament (ALL).

In a similar method, the device may be positioned such that the horizontal and/or vertical arms are placed between layers of the anterior longitudinal ligament (ALL) or between layers of the posterior longitudinal ligament (PLL), Slits could be made partially through either the anterior or posterior longitudinal ligament and the arms of the device could be placed between layers of either the anterior or posterior longitudinal ligament. After positioning the horizontal and/or vertical arms between layers of the anterior or posterior longitudinal ligament, anchors, such as staples, sutures, or screws, could be used to attach the horizontal and/or vertical arms to the surrounding anatomy.

Similarly, the device may be positioned such that the horizontal arms are placed between layers of the anulus fibrosus (AF). Slits in the anulus fibrosus can be made and the horizontal arms may be inserted beneath at least the outer layer of the anulus fibrosus. After positioning the horizontal arms between layers of the anulus fibrosus, anchors, such as staples or sutures, could be used to attach the horizontal arms to the anulus fibrosus.

The device may also be positioned on the outside of the tendon or ligament, i.e., posterior to the posterior longitudinal ligament or anterior to the anterior longitudinal ligament. Where the device is positioned on the outside of the tendon or ligament, an anti-adhesion cover can be positioned over the mesh device to prevent adhesions from forming. Anti-adhesion components may be made from materials that are unlikely to form adhesions with the nerves, such as Gortex, autograft fascia, or allograft fascia. Staples, sutures, or other fastening elements can be used to fasten the horizontal arms of the device and the ligament (posterior or anterior, depending on the location) to the anulus fibrosus.

Although the foregoing invention has, for the purposes of clarity and understanding, been described in some detail by way of illustration and example, it will be obvious that certain changes and modifications may be practiced which will still fall within the scope of the appended claims.

1. A method for fixing a defect in the anulus fibrosus of an intervertebral disc of a patient, the intervertebral disc being located between an upper and a lower vertebrae, comprising the steps of:
providing a device comprising:
a horizontal component having first and second ends, a middle region, and a length; and
first and second vertical components extending from the middle region of the horizontal component, each of the first and second vertical components having a width and an end, wherein the length of the horizontal component is longer than the width of each of the first and second vertical components;
positioning the middle region of the horizontal component over the defect in the anulus fibrosus and the first and second ends of the horizontal component beneath a layer of either a posterior longitudinal ligament or a anterior longitudinal ligament;
attaching the first vertical component to the upper vertebra; and
attaching the second vertical component to the lower vertebra.

2. The method of claim 1, wherein the horizontal component is positioned beyond at least an outer layer of the anulus fibrosus.

3. The method of claim 1, wherein the horizontal component is positioned beyond the innermost layer of anulus fibrosus.

4. The method of claim 1, wherein the horizontal component is positioned between adjacent layers of anulus fibrosus.

5. The method of claim 1, wherein the horizontal component is positioned on the exterior of the anulus fibrosus.

6. The method of claim 1, further comprising the step of attaching the horizontal component to the anulus fibrosus with at least one fixation device.

7. The method of claim 6, wherein the at least one fixation device is a staple.

8. The method of claim 1, further comprising the step of attaching the horizontal component to the anulus fibrosus with first and second fixation devices attached to the horizontal component and anulus fibrosus on either side of the defect.

9. The method of claim 1, further comprising the step of locating a growth promoting component within the defect.

10. The method of claim 9, wherein the growth-promoting component is made from a material selected from the group consisting of allograft tissue, xenograft tissue, collagen-soaked BMP sponges, and autograft material.

11. The method of claim 10, wherein the allograft tissue is selected from the group consisting of fascia, tendon, and anulus fibrosus.

12. The method of claim 10, wherein the xenograft tissue is porcine intestinal sub-mucosa.

13. The method of claim 1, wherein the first vertical component is attached to the upper vertebra by inserting the first vertical component into a hole in the upper vertebra and wherein the second vertical component is attached to the lower vertebra by inserting the second vertical component into a hole in the lower vertebra.

14. The method of claim 13, wherein the first vertical component is attached to the upper vertebra by inserting a first interference screw into the upper vertebra adjacent the first vertical component and wherein the second vertical component is attached to the lower vertebra by inserting a second interference screw into the lower vertebra adjacent the second vertical component.

15. The method of claim 13, further comprising the step of injecting a fixation material into the holes in the upper and lower vertebrae.

16. The method of claim 15, wherein the fixation material is an in-situ curing polymer.

17. The method of claim 13, further comprising inserting a plug into the hole, wherein the plug is made from a material that facilitates bone ingrowth.

18. The method of claim 17, wherein the material that facilitates bone ingrowth is selected from the group consisting of allograft bone, hydroxyapatite, ceramic, and BMP-soaked collagen sponges.

19. The method of claim 1, wherein the first and second vertical components each have an opening located near a distal end of the and second vertical components, and further comprising the steps of:
inserting a first suture through the opening in the first vertical component such that an enlarged proximal end of the first suture does not pass through the opening;
anchoring a distal end of the first suture to the upper vertebra;
inserting a second suture through the opening in the second vertical component such that an enlarged proximal end of the second suture does not pass through the opening; and
anchoring a distal end of the second suture to the lower vertebra.

20. The method of claim 1, further comprising the step of inserting a nucleus replacement into intervertebral disc.
